(19) United States
(12) Patent Application Publication
(10) Pub. No.: US 2024/0207477 A1
(43) Pub. Date: Jun. 27, 2024

(54) PEROXIDE-ENHANCED GERMICIDAL IRRADIATION FOR THE TREATMENT OF AIRBORNE AND SURFACE-ASSOCIATED CONTAMINANTS

(71) Applicant: THE REGENTS OF THE UNIVERSITY OF COLORADO, A BODY CORPORATE, Denver, CO (US)

(72) Inventors: Mark HERNANDEZ, Denver, CO (US); Emmalee BIESIADA, Denver, CO (US)

(21) Appl. No.: 18/573,907

(22) PCT Filed: Jun. 27, 2022
(86) PCT No.: PCT/US2022/035156

§ 371 Date: Dec. 22, 2023

Related U.S. Application Data
(60) Provisional application No. 63/214,919, filed on Jun. 25, 2021.

(51) Int. Cl. A61L 9/04; A61L 2/20; A61L 9/12; A61L 9/20

(52) U.S. Cl. CPC A61L 9/046; A61L 2/208; A61L 9/12; A61L 9/20; A61L 2202/11; A61L 2209/12; A61L 2209/16; A61L 2209/211

(57) ABSTRACT

Described herein are systems and methods for treatment of various airborne or surface-associated contaminants in which UV aerosol technology is combined with the use of hydrogen peroxide vapor or aerosol to create potent airborne concentrations of reactive oxygen-containing radicals that react with the airborne or surface-associated contaminants to inactivate, disinfect, oxidize, rearrange, ablate and/or otherwise treat these materials and thereby achieve disinfection of indoor air and/or surfaces. This technology is referred to herein as peroxide-enhanced germicidal irradiation (PEGI).

### CROSS-REFERENCE TO RELATED APPLICATION(S)

The present application claims priority under 35 U.S.C. § 119(e) to U.S. Provisional Patent Application No. 63/214,919, filed Jun. 25, 2021, the entirety of which is hereby incorporated by reference.

### TECHNICAL FIELD

The present disclosure relates to methods and systems for treatment of contaminants, including both airborne contaminants and surface-associated contaminants. More specifically, this disclosure describes methods and systems utilizing hydrogen peroxide vapors and/or aerosol droplets activated by UV irradiation to oxidize various types of contaminants, whether airborne or located on a surface.

### BACKGROUND

In urban environments, many people spend in excess of 90% of their time indoors where air must be conditioned and exposures to airborne microbes is markedly higher than immediately outdoors. Thus, the sanitation of indoor air is gaining increased attention as a public health and infrastructure priority, particularly in high density public buildings and health care settings. However, existing technologies for sanitation of indoor air fall short of providing an effective, comprehensive, adaptable, safe, economical, and simplified solution.

Generally speaking, use of chemical biocides for aerosol disinfection or HVAC system cleaning is not a practical solution for the sanitation of indoor air because of associated environmental health risks, and has been discouraged by the World Health Organization.

Filtration and ultraviolet (UV) irradiation remain among the few economically viable indoor air treatment alternatives for larger structures. However, while common germicidal wavelengths of UV (above 240 nm) has been used to disinfect indoor air, the effectiveness of such UV light is limited to its “line of sight” which cannot be incident or reflected on occupants because of human health concerns, including skin and eye cancer. Additionally, high efficiency filtration remains prohibitively expensive for most urban building stocks. Furthermore, UV aerosol disinfection has variable efficacy based on environmental conditions (e.g., relative humidity (RH)), and commercial UV system rely on bulky mercury lamps, the contents of which are toxic and represent handling and disposal hazards. Also, UV alone is not effective against microbial toxins and allergens.

Some current technology for HVAC disinfection employs fumigation or conventional ultraviolet germicidal irradiation (UVGI). However, in addition to relying on the same bulky and potentially hazardous UV equipment discussed above, UVGI has no effect on airborne allergens.

Similar issues exist with respect to disinfecting solid surfaces, in that existing disinfection methods for solid surfaces may be costly, inefficient, and/or pose health and environmental risks.

Accordingly, a need exists for improved sanitation systems and methods.

### SUMMARY

This Summary is provided to introduce a selection of concepts in a simplified form that are further described below in the Detailed Description. This Summary, and the foregoing Background, is not intended to identify key aspects or essential aspects of the claimed subject matter. Moreover, this Summary is not intended for use as an aid in determining the scope of the claimed subject matter.

In some embodiments, a method for treatment of contaminants is described, the method generally including the steps of flowing hydrogen peroxide vapor or aerosol into an enclosed space; irradiating the hydrogen peroxide vapor or aerosol with ultraviolet light such that the interaction between the hydrogen peroxide vapor or aerosol and the ultraviolet light produces reactive oxygen-containing radicals; and subjecting contaminants located within the enclosed space to the reactive oxygen-containing radicals to thereby inactivate, disinfect, and or/ablate the contaminants.

In some embodiments, a system for treatment of contaminants is described, the system generally including a source of hydrogen peroxide configured to introduce hydrogen peroxide vapor or aerosol into an enclosed space; and an ultraviolet light source positioned within the enclosed space and configured to irradiate the hydrogen peroxide vapor to form reactive oxygen-containing radicals within the enclosed space.

These and other aspects of the technology described herein will be apparent after consideration of the Detailed Description and Figures herein. It is to be understood, however, that the scope of the claimed subject matter shall be determined by the claims as issued and not by whether given subject matter addresses any or all issues noted in the Background or includes any features or aspects recited in the Summary.

### BRIEF DESCRIPTION OF THE DRAWINGS

FIG. 1 is a flow chart illustrating a method for treatment of contaminants according to various embodiments described herein.

FIG. 2 is a simplified illustration of the reactions and mechanisms of a method for treatment of contaminants according to various embodiments described herein.

FIG. 3 is a schematic illustration of a system for use in treatment of contaminants according to various embodiments described herein.

FIG. 4A is a graph illustrating results of UVGI inactivation response for airborne contaminants.

FIG. 4B is a graph illustrating results of PEGI and UVGI inactivation response at low relative humidity for airborne contaminants.

FIG. 4C is a graph illustrating results of PEGI and UVGI inactivation response at high relative humidity for airborne contaminants.

FIG. 4D is a graph illustrating the combination of the data from FIGS. 4A-4C.

FIG. 5 is a graph illustrating the results of PEGI inactivation response at various concentrations for airborne contaminants.

FIG. 6A is a graph illustrating the results of PEGI and UVGI inactivation of airborne contaminants at UV wavelength of 222 nm, 1 m³ enclosed space volume, and 25% relative humidity.

FIG. 6B is a graph illustrating the results of PEGI and UVGI inactivation of airborne contaminants at UV wavelength of 254 nm, 9 m³ enclosed space volume, and 60% relative humidity.

FIG. 7A is a graph illustrating the results of PEGI and UVGI inactivation of surface-associated contaminants on artificial leather.

FIG. 7B is a graph illustrating the results of PEGI and UVGI inactivation of surface-associated contaminants on polycarbonate.

### DETAILED DESCRIPTION

Described herein are systems and methods for treatment of various contaminants in which UV aerosol technology is combined with the use of hydrogen peroxide vapor or aerosol to create potent airborne concentrations of hydroxyl and/or other oxidizing radicals that react with contaminants, whether airborne or surface-associated, to inactivate, disinfect, oxidize (partially or fully), rearrange, and or/ablate these materials and thereby achieve sterilization, disinfection and/or purification of, e.g., indoor air and surfaces of various materials. This technology is referred to herein as peroxide-enhanced germicidal irradiation (PEGI). PEGI may include, but is not limited to, use of all UV wavelengths (>200 nm) that activate airborne hydrogen peroxide to form reactive oxygen-containing species (including but not limited to hydroxyl radicals) in contained environments. The methods and systems described herein generally relate to non-bulk liquid environments, and as such, should not be confused with methods and systems for treatment of, e.g., drinking water or wastewater.

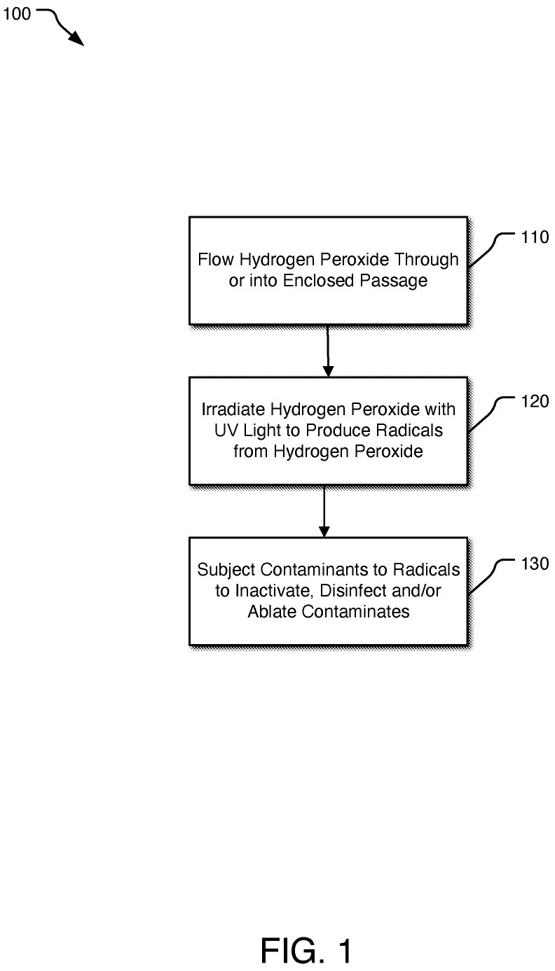

With reference to FIG. 1, a method 100 for treating contaminants according to some embodiments described herein may include a step 110 of flowing hydrogen peroxide vapor or aerosol through or into an enclosed space, a step 120 of irradiating the hydrogen peroxide vapor with ultraviolet light such that the interaction between the hydrogen peroxide vapor and the ultraviolet light produces a reactive oxygen-containing species (e.g., hydroxyl radicals), and a step 130 of subjecting contaminants to the reactive species to thereby inactivate, disinfect, oxidize, rearrange, and or/ablate the contaminants.

In step 110, hydrogen peroxide vapor or aerosol is flowed into an enclosed space. In some embodiments, the term “enclosed space” is used herein to denote any confined area, indoor room, passageway, or the like. In some embodiments, the enclosed space is used for the passage and/or circulation of air throughout a structure or portion thereof. In one non-limiting example, the enclosed space is a portion or all of a heating, ventilation, and air conditioning (HVAC) system, such as a duct or series of ducts of a HVAC system.

The enclosed space may include surfaces and/or have contained therein surfaces which may have contaminants located thereon. For example, the enclosed space may be defined by a series of walls, and the walls may have contaminants disposed thereon. In one specific though non-limiting example where the enclosed space includes ducts of a HVAC systems, the interior surfaces of the walls of the ducts may have contaminants disposed thereon. In other embodiments, surfaces that do not make up part of the enclosed space may be located within the enclosed space and have contaminants located thereon. For example, any of a variety of different objects may be located within the enclosed space, and one or more surfaces (e.g., exterior surfaces) of these objects may have contaminants disposed thereon. In one non-limiting example, a table, chair (including a wheelchair), countertop or any other architectural appurtenances may be disposed within the enclosed space and have contaminants disposed on the surfaces thereof.

Hydrogen peroxide vapor or aerosol can be flowed or otherwise introduced into the enclosed space using any suitable techniques and using any suitable equipment. In some embodiments, a source of hydrogen peroxide vapor or aerosol is provided proximate the enclosed space and includes means for passing hydrogen peroxide vapor or aerosol from the hydrogen peroxide source to the enclosed space, such as through the use of a pump and tubing that may run from the hydrogen peroxide source and the enclosed space. The enclosed space may include a port, valve, or other type of opening suitable for use in introducing hydrogen peroxide vapor or aerosol into the interior of the enclosed space. In other embodiments, one or more fans may be used to propel hydrogen peroxide vapor or aerosol into the enclosed space.

In some embodiments, flow control equipment is provided so that the amount of hydrogen peroxide vapor or aerosol introduced into the enclosed space is precisely controllable. In some embodiments, the concentration of the hydrogen peroxide introduced into the enclosed space is controlled via the flow control equipment. For example, flow control equipment can be used to ensure that the concentration of hydrogen peroxide introduced into the enclosed space does not exceed health regulations set by local, state, and/or federal agencies. In some embodiments, the amount of hydrogen peroxide introduced into the enclosed space is controlled to be less than 1 ppmv such that the amount of hydrogen peroxide used complies with OSHA standards. In some embodiments, hydrogen peroxide is introduced at an amount less than 350 ppb, less than 35 ppb, or less than 3 ppb, though any other regulation-compliant amounts can also be used.

In some embodiments where health regulations, human safety, etc., may not apply, the concentration of hydrogen peroxide may be higher than 1 ppm. For example, in unoccupied spaces, the hydrogen peroxide concentration used can be higher than 1 ppm, and in some cases, substantially higher than 1 ppm. Because the systems and methods described herein rapidly accelerate the decontamination effect as compared to, e.g., hydrogen peroxide alone or UV light alone, use of concentrations above 1 ppm, such as in a fumigation scenario, can result in expedient decontamination of unoccupied spaces. The same holds true for UV intensities used in the methods and systems described herein. While UV light intensity may be limited by health regulations in treatment of occupied spaces, such limitations on UV light intensity may be relaxed or entirely removed in unoccupied spaces.

As noted above, the hydrogen peroxide is introduced into the enclosed space in the form of vapor or aerosol droplets or both. As such, the means for introducing hydrogen peroxide into the enclosed space may include means for providing the hydrogen peroxide in a vapor form and/or aerosol form. For example, if a hydrogen peroxide source located proximate the enclosed space stores hydrogen peroxide in a liquid form, then the means for passing the hydrogen peroxide from the source to the enclosed space may include equipment that vaporizes, nebulizes or otherwise converts the liquid hydrogen peroxide to a vapor and/or aerosol droplet form. The hydrogen peroxide may, in some embodiments, be mixed with other components. In one non-limiting example, hydrogen peroxide is mixed with other chemicals that aid in stabilizing the hydrogen peroxide or otherwise affect is reactivity.

Once introduced into the enclosed space, the hydrogen peroxide vapor or aerosol may flow around the enclosed space, including through the enclosed space if there is a direction of air flow already provided within the enclosed space. For example, in the case of introducing hydrogen peroxide vapor or aerosol into an HVAC system, the hydrogen peroxide may begin to flow in the direction of air flow through the HVAC system. The hydrogen peroxide will also begin to mix and/or become interspersed with air and any other constituents in the air (e.g., contaminants) flowing through the HVAC system. The hydrogen peroxide will also contact any surfaces located within the enclosed space, including the interior surfaces of the walls defining the enclosed space.

In order to promote movement of the hydrogen peroxide vapor or aerosol within, around, and/or through the enclosed space, one or more fans or other means of propelling/moving the hydrogen peroxide may be provided. The one or more fans or other means for propelling moving the hydrogen peroxide may also be used to encourage contact of the hydrogen peroxide (or radicals formed therefrom as discussed in greater detail below) with any surfaces located within the enclosed space.

In some embodiments, the hydrogen peroxide vapor or aerosol introduced into the enclosed space is allowed to reside within the enclosed space for a period of time before irradiating the hydrogen peroxide with UV light as per step 120 described in greater detail below. During this time period after hydrogen peroxide is introduced into the enclosed space but prior to irradiating the hydrogen peroxide with UV light, the hydrogen peroxide may contact contaminants located within the enclosed space (whether airborne or surface-associated). The specific amount of time during which the hydrogen peroxide is maintained in the enclosed space prior to irradiation with UV light is generally not limited. In some embodiments, the time period may be a matter of minutes or hours (e.g., 5 minutes, 15 minutes, 30 minutes, 1 hour, 2 hours, 3 hours, etc.), while in other embodiments the time period may be longer such as in the order of a half day, a fully day, or multiple days.

In step 120, the hydrogen peroxide vapor or aerosol introduced into the enclosed space is irradiated with ultraviolet light in order to produce reactive oxygen-containing radicals from the hydrogen peroxide. Any suitable manner of providing ultraviolet irradiation using any suitable equipment can be used to carry out step 120. In some embodiments, a UV light source is provided inside the enclosed space such that the hydrogen peroxide introduced into the enclosed space can be irradiated by the UV light.

In some embodiments, UV light is provided by locating a UV light source inside the enclosed space. The UV light source is not limited, and in some embodiments, may be, for example, one or more UV lamps or one or more UV light emitting diodes, which can emit either a narrow-band or broad-band of UV wavelengths. Other sources of UV light can also be used, such as Hg lamps, KrCl sources and other excimer or solid state UV sources. In some embodiments where air flows in a direction through the enclosed space, the UV light source can be located downstream of the location where hydrogen peroxide is introduced into the enclosed space. In this manner, there is a higher probability that all hydrogen peroxide vapor or aerosol will flow downstream and past the UV light source such that most or all hydrogen peroxide introduced into the enclosed space will be converted to reactive oxygen-containing radicals.

The specific wavelength of the UV light used in step 120 is generally not limited. In some embodiments, the wavelength is in the range of from about 200 nm to about 280 nm. In some embodiments, two or more UV light sources may be provided, each UV light source providing a different UV wavelengths. For example, in some embodiments, a first UV light emitting diode (or series of UV LEDs) emitting UV light at a wavelength of 260 nm is provided followed by a second UV LED (or series of UV LEDs) emitting UV light at a wavelength of about 280 nm. Hydrogen peroxide flowing through the enclosed space and past the UV light sources will be first be irradiated by 260 nm UV light and then 280 nm UV light. Any number of UV light sources providing any combination of UV wavelengths in any sequence can be used.

The specific location of the UV light source within the enclosed space is generally not limited. In some embodiments, the UV light sources are disposed on an interior surface of a wall of the enclosed space such that UV light is projected into the enclosed space from the sides of the enclosed space. In other embodiments, the UV light sources are positioned in the middle of the enclosed space, such as through the use of a stand or pole extending from a wall of the enclosed space into the interior space of the enclosed space and having a UV light source located at the terminal end of the stand or pole. In such embodiments, UV light may be projected in all or nearly all directions from the UV light source. UV light sources used in the methods and systems described herein may also be mobile UV light sources such that the location of the UV light source can be manually or automatically changed or adjusted, either before, during or after the methods described herein. Such mobile UV light sources may be especially well suited for configurations deployed to treat surfaces of health care settings.

In more static environments, additional steps and/or equipment may be used in order to promote movement of hydrogen peroxide through the enclosed space and past UV lights, and to promote interaction between formed radicals and contaminants, whether airborne or surface-associated (as discussed in greater detail below). For example, fans, impellers and the like may be used to promote flow and mixing within the enclosed space.

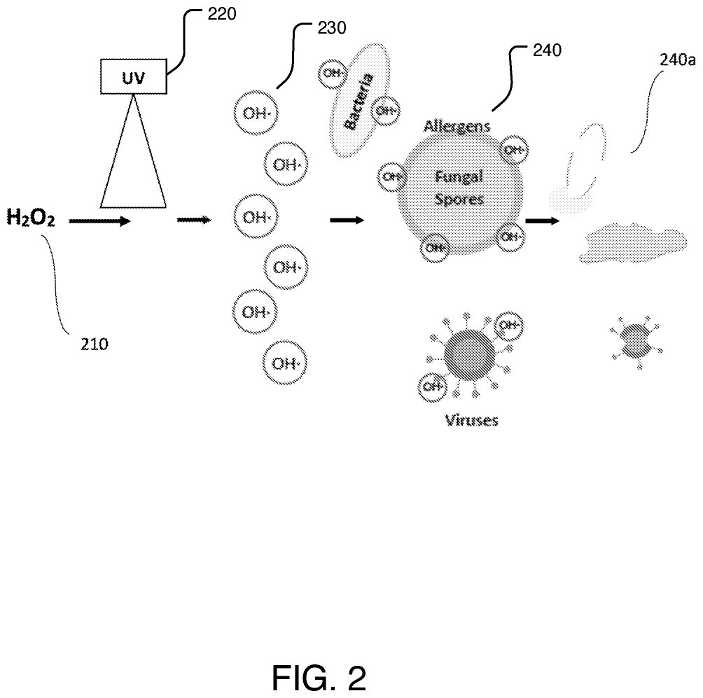

In step 130, the radicals created in step 120 interact with contaminants located anywhere within the enclosed space, including both airborne and surface-associated contaminants. These interactions lead to inactivating, disinfecting, ablating, and/or otherwise treating the contaminants. FIG. 2 provides an illustration of this process in which hydrogen peroxide 210 at trace levels (i.e., ppb) is subjected to UV light 220 to thereby form hydroxyl radicals 230, followed by the hydroxyl radicals 230 interacting with contaminants 240 (e.g., bacteria, fungal spores, viruses, etc.) to inactive, disinfect, rearrange, oxidize and/or ablate the contaminants 240. Once inactivated, disinfected, rearranged, oxidized and/or ablated, the contaminants 240 become treated contaminants 240a that no longer retain the properties that pose a threat to human health or pose a reduced threat to human health. As such, the term “treated” may generally be used to encompass all of inactivating, disinfecting, rearranging, oxidizing, ablating or any other means for altering the contaminants such that the contaminants no longer retain the properties that pose a threat to human health or pose a reduced threat to human health.

As discussed herein, the radicals created in step 120 are used to inactivate, disinfect, ablate and/or otherwise treat contaminants. The term contaminants used herein is intended to have broad scope, and may include (but is not limited to) bioaerosols generated by human activity, infectious microbes, allergenic agents, microbial toxins, microbial allergens, bacteria, fungal spores, viruses, their component parts and any other germicidal agents. The contaminants may include biological and/or non-biological material. With respect to surface-associated contaminants, this may include (but is not limited to) any of the previously described types of contaminants which may be permanently or temporarily associated with a surface. The systems and methods described herein may be applied to aerosols as well as particulate matter on surfaces as fomites and biofilms, regardless of how long they have been there, or will be there, or their biological origins, biopolymeric content and inorganic content. In one specific, though non-limiting, embodiment, the contaminant includes CoVID-19.

The radicals created from UV irradiation of hydrogen peroxide may be any reactive oxygen-containing radical generated by or generatable by the interaction of hydrogen peroxide and UV light. The formed radical is, generally speaking, an oxidizing radical. In one non-limiting example, the radical is a hydroxyl radical. However, the radical may be other types besides hydroxyl radicals. Non-limiting examples of other radicals that may be used include superoxides, HO₂—, HO₂·—, and O₂·—. Generally speaking, the phrase “reactive oxygen-containing radical” and versions thereof should be interpreted to be inclusive of all downstream reactive oxygen-containing radicals generatable from the irradiation of hydrogen peroxide with UV light.

While method 100 has been illustrated and described as sequentially providing hydrogen peroxide into an enclosed space followed by irradiating the hydrogen peroxide with UV light, it should be appreciated that the order of steps can be altered and/or performed sequentially. For example, hydrogen peroxide may be introduced into the enclosed space at the same time as irradiating the hydrogen peroxide with UV light. It may also be possible to irradiate hydrogen peroxide with UV light prior to introducing the hydrogen peroxide into the enclosed space. In such embodiments, the material introduced into the enclosed space may be a combination of hydrogen peroxide and already formed reactive oxygen-containing radicals.

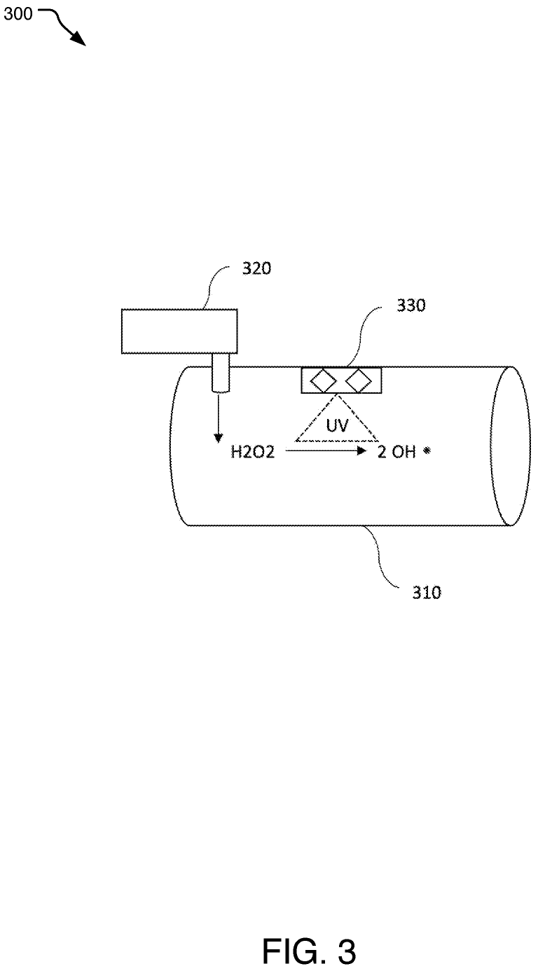

FIG. 3 provides a schematic illustration of system 300 suitable for use in carrying out embodiments of the method described herein. System 300 is generally installed or used in conjunction with an enclosed space 310, which in FIG. 3 is represented by a cylindrical passage through which air may flow (e.g., from left to right as shown in FIG. 3), and which in some embodiments may be a section of an HVAC passage, transportation vehicle, super- or sub-terranean tunnel, or any other indoor building feature or appurtenance. While FIG. 3 shows the enclosed space 310 having a cylindrical geometry, it should be appreciated that the specific geometry and dimensions of the enclosed space 310 are not limited.

In some embodiments, at least a portion of the interior surface of the enclosed space 310 is reflective. For example, the entire interior surface may be reflective, or the portion of the interior surface proximate the UV light source 330 may be reflective. Providing at least a portion of the interior surface with reflective properties can help to ensure that UV light emitted from the UV light source 330 reflects back and forth within the enclosed space to thereby increase the chance of interaction between UV light and the hydrogen peroxide to produce the desire radicals. Any manner of providing a reflective interior surface can be used, including applying a reflective coating or using a material that is naturally reflective. The specific material or coating used to provide a reflective surface is also not limited, though in some embodiments, it is preferable that the reflective material or coating be reflective of UV light.

System 300 further includes a hydrogen peroxide source 320 for supplying hydrogen peroxide into the enclosed space 310. As discussed in greater detail above, the specific equipment used for the hydrogen peroxide source 320 is not limited provided that the source 320 is capable of supplying into the enclosed space 310 a controlled amount of hydrogen peroxide vapor or aerosol droplets. Exemplary equipment that may be part of the hydrogen peroxide source 320 includes but is not limited to, piezoelectric distributors, pumps, transport tubing, means for providing hydrogen peroxide in a vapor or aerosol droplet form (e.g., nebulizer), etc. As also shown in FIG. 3 and as discussed previously, the enclosed space 310 may include a port, valve, or other type of entry point so that hydrogen peroxide supplied by hydrogen peroxide source 320 may be introduced into the enclosed space 310.

System 300 further includes a UV light source 330. As discussed previously, the specific equipment used for UV light source 330 is not limited, and may include, for example, one or more UV lamps, one or more UV LEDs, one or more excimer sources, one or more solid state sources, or any other equipment that emits UV light. In an exemplary embodiment shown in FIG. 3, the UV light source 330 includes two UV light sources, each emitting UV light at a different wavelength. The first UV light source, which may be an array of UV LEDs, can emit UV light at, e.g., 260 nm, while the second UV light source, which be an array of UV LEDs, can emit UV light at, e.g., 280 nm. As discussed previously, any combination of UV light source types, wavelengths, number of UV light sources, etc., can be used. Similarly, while FIG. 3 illustrates the UV light source 330 located at a top portion of the enclosed space 310, it should be appreciated that the exact location, orientation, etc., of the UV light source 330 is not limited.

As shown in FIG. 3, system 300 is designed such that air flows through the enclosed space 310 from left to right, hydrogen peroxide source 320 is located at an upstream end of the enclosed space, and UV light source 330 is located downstream of the hydrogen peroxide source 320 entry point. This configuration helps to ensure that all hydrogen peroxide introduced into the enclosed space 310 flows past the UV light source 330, which in turn helps to ensure that all hydrogen peroxide introduced into the enclosed space 310 is converted to radicals (specifically hydroxyl radicals as shown in FIG. 3). However, it should be appreciated that other configurations can also be used, such as where the hydrogen peroxide is introduced into the enclosed space 310 at approximately the same location as the UV light source 320. In more static environments where flow of air does not have as great an effect on the direction of flow of the hydrogen peroxide through the enclosed space 310, the specific location of the hydrogen peroxide source 320 entry point and the UV light source 330 relative to each other may be even less important and open to more design options.

While not shown in FIG. 3, additional equipment can be provided as part of the system 300 as necessary for the specific application of the technology disclosed herein. For examples, fans, impellers or the like can be provided at various locations within the system 300 for promoting the flow hydrogen peroxide and the mixing of hydrogen peroxide with the air to be treated and/or the UV light source.

### EXAMPLES

Experimentation was carried out to determine the bioaerosol inactivation effectiveness in a simulated HVAC environment for PEGI technology as described herein and as compared to a control setting and standard ultraviolet germicidal irradiation (UVGI) (i.e., only UV irradiation is provided, without introduction of hydrogen peroxide). The system set up was similar to that shown in FIG. 3, with the control system providing no UV or hydrogen peroxide treatment, UVGI using only UV light treatment (tested at various wavelengths and using different combinations of UV light sources), and PEGI using both hydrogen peroxide and UV light treatment (tested at various wavelengths and using different combinations of UV light sources, and also varying hydrogen peroxide concentration). A live airborne bacteria (B. subtilis) was introduced into the model system. Variations in relative humidity were also tested to determine how bioaerosol inactivation is impacted.

In one set of tests designed for analyzing the PEGI model, the concentration of hydrogen peroxide introduced into the system was tested at 350 ppbv, 35 ppbv and 3 ppbv.

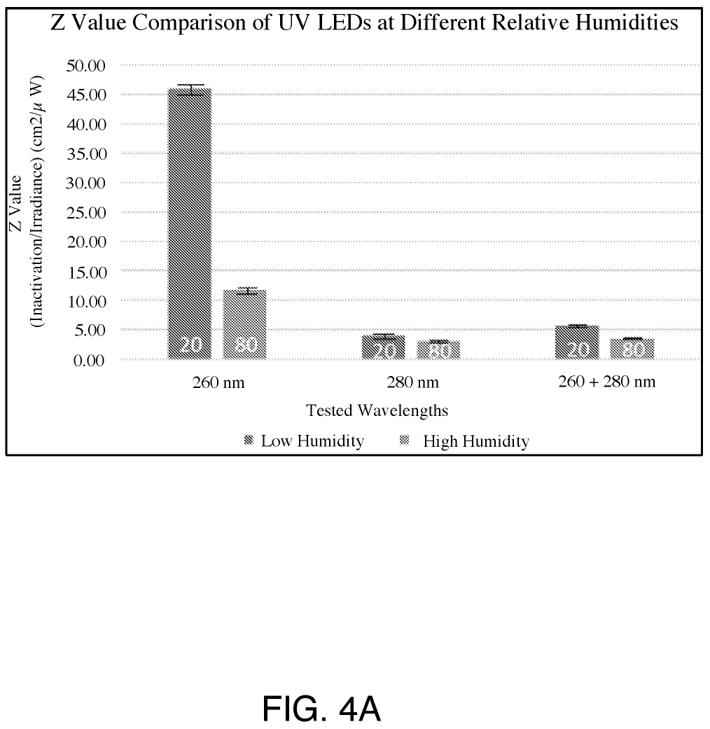

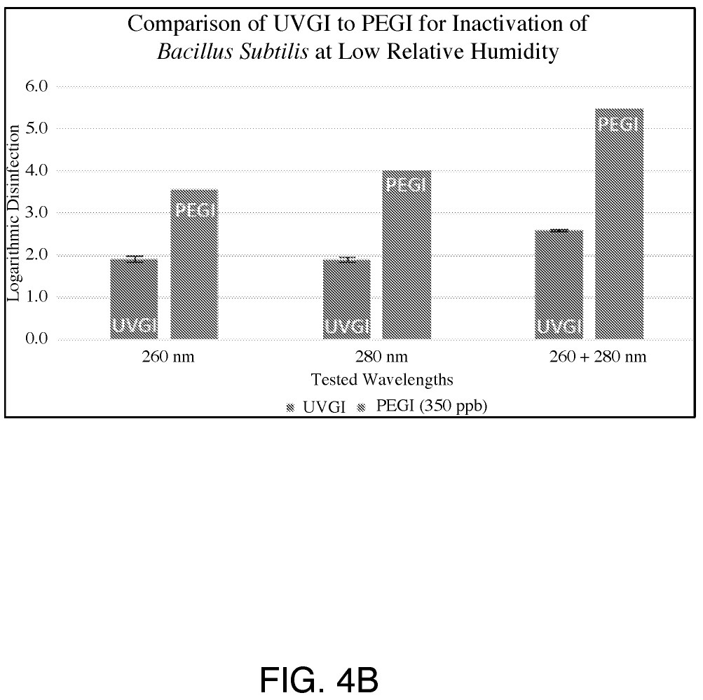

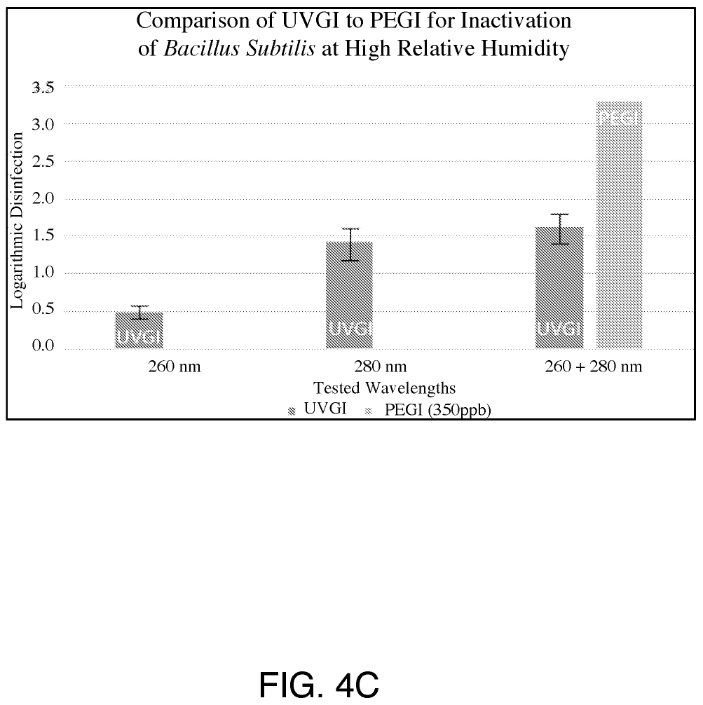

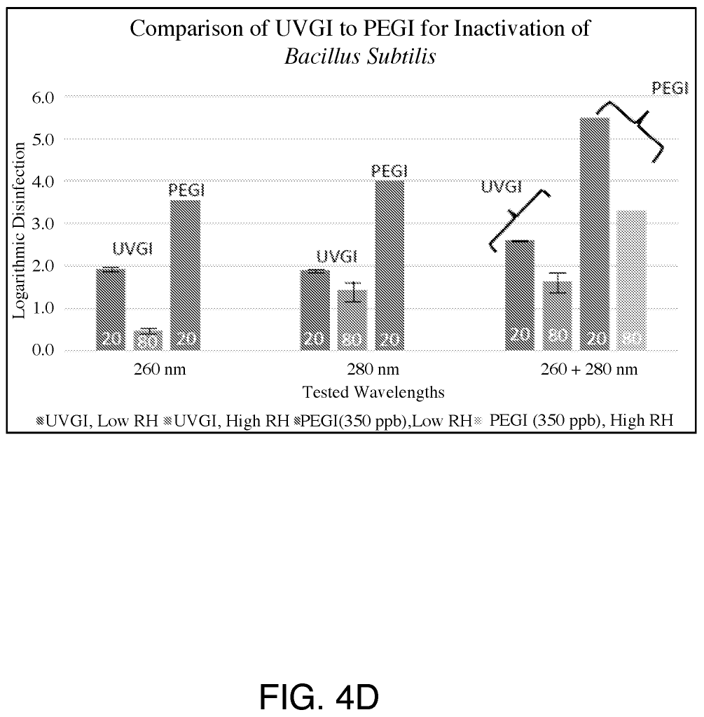

FIG. 4A provides a graph illustrating the results of tests carried out for UVGI inactivation response. UVGI was tested at low (<20%) and high (80-90%) humidity, and at 260 nm wavelength, 280 nm wavelength, and 260+280 wavelength. As shown in FIG. 4A, the “Z value” (inactivation normalized by fluence) for UVGI at 260 nm performed the worst (i.e., 10% of results achieved at 280 nm).

FIG. 4B provides a graph illustrating the performance of PEGI compared to UVGI for airborne disinfection at low relative humidity and using a 350 ppbv concentration of hydrogen peroxide in the PEGI method. As shown in FIG. 4B, PEGI performed better at all wavelengths and wavelength combinations tested.

FIG. 4C provides a graph illustrating the performance of PEGI compared to UVGI for airborne disinfection at high relative humidity and using a 350 ppbv concentration of hydrogen peroxide in the PEGI method. As shown in FIG. 4C, PEGI performed better at the 260+280 nm wavelength (PEGI not tested at 260 nm individually or 280 nm individually at high relative humidity).

FIG. 4D combines results from FIGS. 4A-4C to provide an overall comparison of the testing. At 260 nm, PEGI at low RH performed better than UVGI at low or high RH; at 280 nm, PEGI at low RH performed better than UVGI at low or high RH; and at 260+280 nm, PEGI at low RH performed best, while PEGI at low or high RH perform better than UVGI at low or high RH.

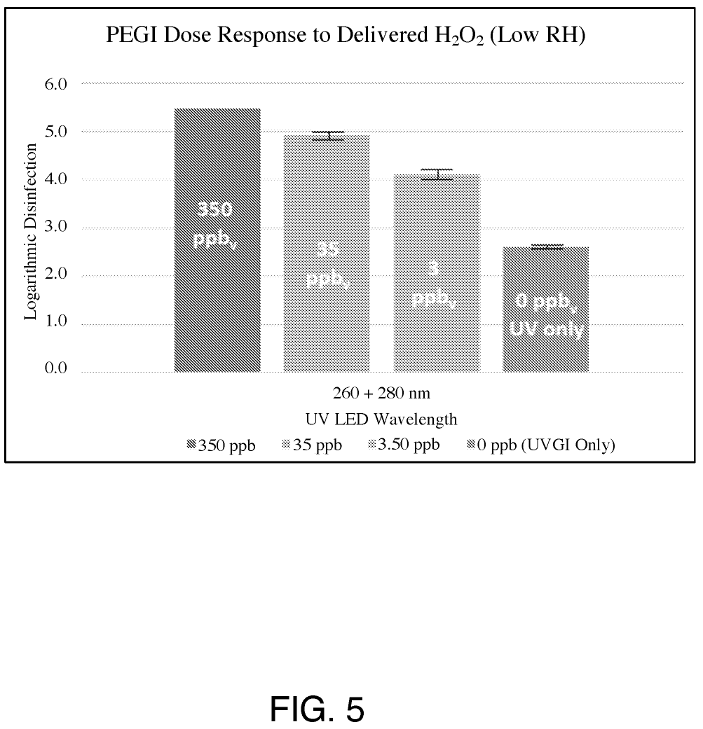

In FIG. 5, PEGI is analyzed at various concentrations using 260+280 nm. The results show the best performance for PEGI at 350 ppb, but still a marked improvement of PEGI over UV only even at concentrations as low as 3 ppb.

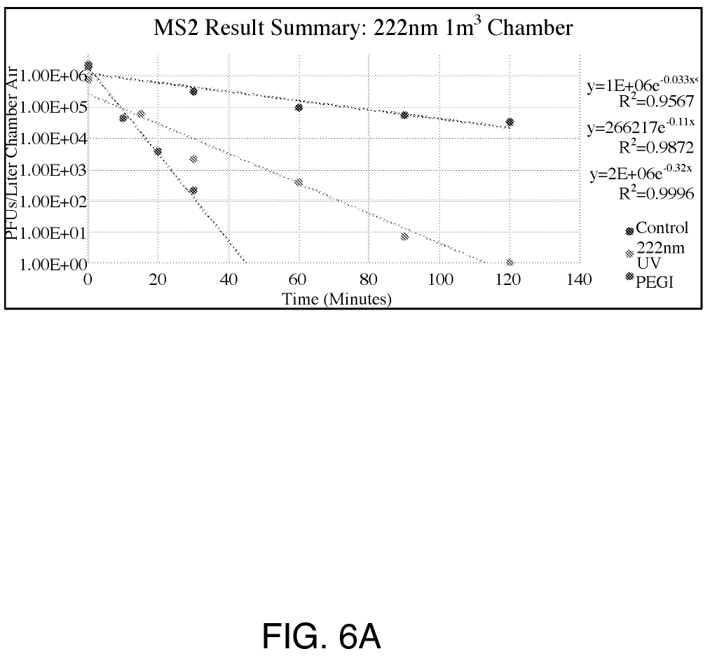

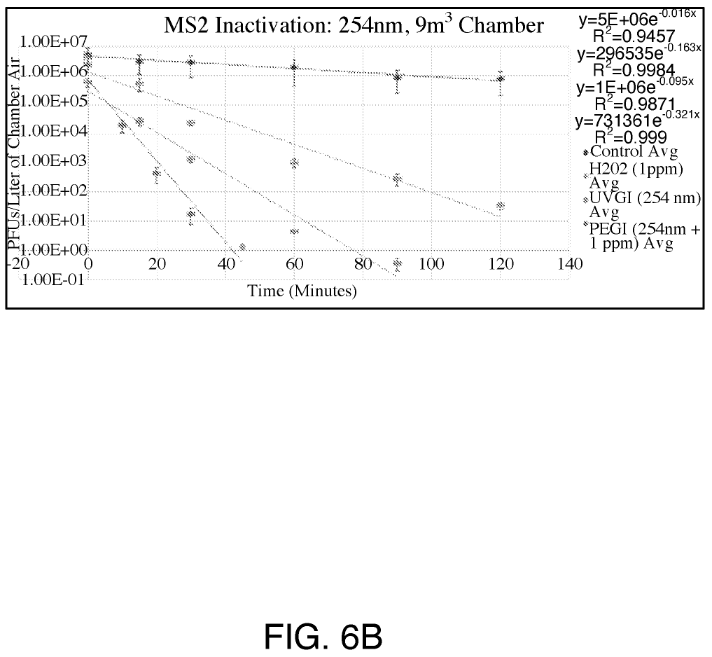

FIG. 6A provides a graph illustrating the performance of PEGI compared to UVGI for an enclosed space volume of 1 m³ using UV wavelength of 222 nm and relative humidity of 25%. The airborne contaminant provided in the enclosed space was Bacteriophage MS2 virus. As shown in FIG. 6A, the reduction in MS2 concentration was substantially faster for PEGI as compared to either UVGI or control.

FIG. 6B provides a graph illustrating the performance of PEGI compared to UVGI for an enclosed space volume of 9 m³ using UV wavelength of 254 nm and relative humidity of 60%. The airborne contaminant provided in the enclosed space was Bacteriophage MS2 virus. As shown in FIG. 6B, the reduction in MS2 concentration was substantially faster for PEGI as compared to UVGI, hydrogen peroxide alone, or control.

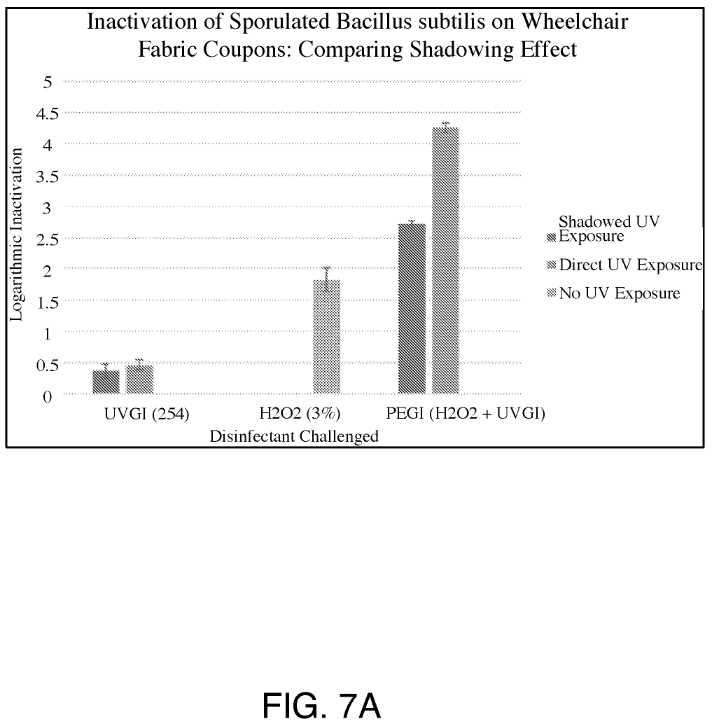

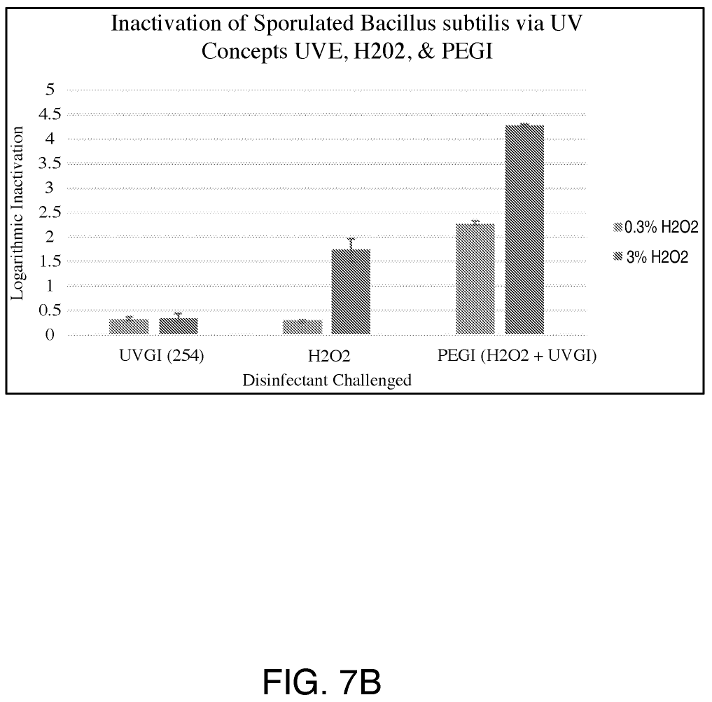

FIG. 7A provides a graph illustrating the performance of PEGI compared to UVGI for inactivating surface-associated contaminants inside an enclosed space volume. The surface-associated contaminant was Sporulated Bacillus spores disposed on coupons of artificial leather located within a 5 m³ enclosed space. The UV wavelength was 254 nm, and both direct UV exposure and shadowed UV exposure were tested. As shown in FIG. 7A, PEGI outperformed UVGI and hydrogen peroxide only for both direct and shadow UV exposure.

FIG. 7B provides a graph illustrating the performance of PEGI compared to UVGI for inactivating surface-associated contaminants inside an enclosed space volume. The surface-associated contaminant was Sporulated Bacillus spores disposed on coupons of polycarbonate located within a 5 m³ enclosed space. The UV wavelength was 254 nm, and different hydrogen peroxide concentrations (0.3% and 3.0%) were tested. As shown in FIG. 7B, PEGI outperformed UVGI and hydrogen peroxide only at both H₂O₂ concentrations.

Experimental Summary: In a dose-response paradigm, airborne and surface-associated contaminant disinfection induced by UV-LEDs was significantly enhanced by trace amounts hydrogen peroxide regardless of humidity levels. Where UV-LED exposures induced contaminant culturability losses on the order of 10² CFU/m³, co-exposure to H₂O₂ levels as low as 90 ppbv, increased inactivation response over >10⁵ CFU/m³. At these levels, H₂O₂ vapor alone could not induce any measurable disinfection response. These results suggest that advanced oxidation processes enabled by UV-LEDs offer new aerosol disinfection alternatives for the built environment.

A repeatable contaminant disinfection response can now be observed from airborne Bacillus subtilis cells, when challenged with trace amounts of hydrogen peroxide in the presence of 260 nm light in environmentally controlled chambers. At low relative humidity (20% RH), PEGI was at least 10³ times more effective than UVGI aerosol disinfection alone; and, at high relative humidity (80%), PEGI was at least 10^{1.5} more effective than UVGI alone. The synergistic disinfection responses observed occurred in less than 5 seconds of PEGI exposure, at room temperature where UV LEDs activated less than 0.5 ppm of H₂O₂ vapor in a mock HVAC duct.

From the foregoing, it will be appreciated that specific embodiments of the invention have been described herein for purposes of illustration, but that various modifications may be made without deviating from the scope of the invention. Accordingly, the invention is not limited except as by the appended claims.

Although the technology has been described in language that is specific to certain structures and materials, it is to be understood that the invention defined in the appended claims is not necessarily limited to the specific structures and materials described. Rather, the specific aspects are described as forms of implementing the claimed invention. Because many embodiments of the invention can be practiced without departing from the spirit and scope of the invention, the invention resides in the claims hereinafter appended.

Unless otherwise indicated, all number or expressions, such as those expressing dimensions, physical characteristics, etc., used in the specification (other than the claims) are understood as modified in all instances by the term “approximately”. At the very least, and not as an attempt to limit the application of the doctrine of equivalents to the claims, each numerical parameter recited in the specification or claims which is modified by the term “approximately” should at least be construed in light of the number of recited significant digits and by applying rounding techniques. Moreover, all ranges disclosed herein are to be understood to encompass and provide support for claims that recite any and all sub-ranges or any and all individual values subsumed therein. For example, a stated range of 1 to 10 should be considered to include and provide support for claims that recite any and all sub-ranges or individual values that are between and/or inclusive of the minimum value of 1 and the maximum value of 10; that is, all sub-ranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

I/We claim:

1. A method for treatment of contaminants comprising:
flowing hydrogen peroxide vapor or aerosol into an enclosed space;
irradiating the hydrogen peroxide vapor or aerosol with ultraviolet light such that the interaction between the hydrogen peroxide vapor or aerosol and the ultraviolet light produces reactive oxygen-containing radicals; and
subjecting contaminants located within the enclosed space to the reactive oxygen-containing radicals to thereby inactivate, disinfect, oxidize, rearrange and/or ablate the contaminants.

2. The method of claim 1, wherein the enclosed space is a portion of a heating, venting, and air conditioning (HVAC) system.

3. The method of claim 1, wherein flowing the hydrogen peroxide comprising flowing hydrogen peroxide at a concentration of less than 1 ppm.

4. The method of claim 1, wherein irradiating the hydrogen peroxide vapor or aerosol with ultraviolet light comprises irradiating the hydrogen peroxide vapor or aerosol with ultraviolet light having a wavelength of from about 200 nm to about 280 nm.

5. The method of claim 1, wherein the contaminants comprise one or more of infectious microbes, allergenic agents, microbial toxins, microbial allergens, bacteria, fungal spores, and viruses.

6. The method of claim 1, wherein the reactive oxygen-containing radicals are hydroxyl radicals.

7. The method of claim 1, wherein the contaminants located within the enclosed space are airborne contaminants.

8. The method of claim 1, wherein the contaminants located within the enclosed space are surface-associated contaminants.

9. The method of claim 1, wherein irradiating the hydrogen peroxide vapor or aerosol with ultraviolet light comprises irradiating the hydrogen peroxide vapor or aerosol with ultraviolet light having a first wavelength and irradiating the hydrogen peroxide vapor or aerosol with ultraviolet light having a second wavelength.

10. A system for treatment of contaminants comprising:
a source of hydrogen peroxide configured to introduce hydrogen peroxide vapor or aerosol into an enclosed space; and
an ultraviolet light source positioned within the enclosed space and configured to irradiate the hydrogen peroxide vapor to form reactive oxygen-containing radicals within the enclosed space.

11. The system of claim 10, wherein the source of hydrogen peroxide is configured to control the amount of hydrogen peroxide vapor or aerosol introduced into the enclosed space such that the concentration hydrogen peroxide introduced into the enclosed space is less than 1 ppm.

12. The system of claim 10, wherein the system further comprises the enclosed space, and the enclosed space is a portion of a heating, venting, and air conditioning (HVAC) system.

13. The system of claim 10, wherein the ultraviolet light source comprises one or more ultraviolet light emitting diodes.

14. The system of claim 10, wherein the system is configured such that the hydrogen peroxide vapor or aerosol flows past the ultraviolet light source.

15. The system of claim 10, wherein the ultraviolet light source is configured to emit ultraviolet light at a wavelength in the range of from about 200 nm to about 280 nm.

16. The system of claim 12, wherein at least a portion of the interior walls of the enclosed space are reflective of ultraviolet light.

17. The system of claim 10, wherein the ultraviolet light source comprises a first ultraviolet light source configured to emit ultraviolet light at a first wavelength and a second ultraviolet light source configured to emit ultraviolet light at a second wavelength different from the first wavelength.

18. The system of claim 12, further comprising an airborne contaminant located within the enclosed space.

19. The system of claim 12, further comprising a surface-associated contaminant located within the enclosed space.

20. The system of claim 10, further comprising means for moving the hydrogen peroxide within the enclosed space and for encouraging interaction between radicals formed from the hydrogen peroxide and contaminants located within the enclosed space.
